(19) United States
(12) Patent Application Publication
(10) Pub. No.: US 2011/0177015 A1
(43) Pub. Date: Jul. 21, 2011

(54) SKIN AND HAIR CARE USING EXTRACT FROM CONDITIONED MEDIUM CULTURED BY MESENCHYMAL STEM CELLS AND OTHER REGENERATIVE CELLS

(76) Inventor: Hymie Friedlander, Kiryat Ono (IL)

(21) Appl. No.: 13/121,462

(22) PCT Filed: Oct. 11, 2009
(86) PCT No.: PCT/IL2009/000954

§ 371 Date: Mar. 29, 2011

Related U.S. Application Data
(60) Provisional application No. 61/102,843, filed on Oct. 5, 2008.

(51) Int. Cl. A61K 8/98; A61K 35/12; A61K 35/16; A61K 38/43; A61Q 5/00; A61Q 7/00; A61P 17/00; A61Q 19/00

(52) U.S. Cl. 424/70.1; 424/520; 424/531; 424/94.1

(57) ABSTRACT

A method and composition for treating dermatological conditions and improving skin condition and helping hair to grow or thicken involves putting mesenchymal stem cells or other cells with regenerative properties into culture medium to induce secretion of beneficial factors such as growth factor, regulatory factors, enzymes, hormones, peptides and lymphokines into the culture medium to create conditioned medium and then extracting either a supernatant or a cell free extract of the conditioned medium to use itself or components thereof alone or with other skin or hair care reagents as a topical ointment or formula to apply to the skin or hair topically for therapeutic and cosmetic purposes.

### PRIORITY INFORMATION

This patent application claims the benefit of and is a continuation-in-part of U.S. Provisional patent application No. 61/102,843, filed Oct. 5, 2008.

### FIELD AND BACKGROUND OF THE INVENTION

The present invention relates to skin care and hair care products and, more particularly, to a method and composition for therapeutic and cosmetic skin care and hair care using extracts from medium conditioned by regenerative cells.

Various dermatological conditions, including but not limited to dermatitis, acne, skin irritation, skin rash and dry skin, affect human skin and there is an obvious need to treat them effectively. In addition, there is a widespread desire for people to retard the effects on their skin from normal aging including by improving skin texture and reducing wrinkles. Protection of the skin from overexposure to ultra violet light and injury to skin from surgical suturing are additional examples in which a need to improve care for the skin is recognized.

With respect to hair, premature or natural hair loss and thinning hair are well known problems that affect both men and women. Various attempts have been made to compose formulae and methods to care for the longevity and quality of the skin, which is an important organ of the body, and to care for the quality and quantity of one's hair, including by enhancing the growth of hair. Such ointments and products have been available for a long time. In addition, numerous products including ointments, creams, gels, powders and other products purport to condition or improve various properties of the skin or hair including their appearance, shine, texture, strength, condition, thickness, smoothness, color, etc.

It would be highly advantageous to have a product for skin care and a product for hair care that has inherent qualities and ingredients that provide active and long lasting care for the skin suffering from skin conditions or improving the youth or other qualities of the skin; and it would be highly advantageous to have a product that improves the qualities of human hair as well as retards or reverses hair loss or thickens thin or thinning hair. It would also be particularly advantageous to have a product or method that would be useful for both skin care and hair care.

### SUMMARY OF THE INVENTION

According to the present invention there is provided a method comprising adding regenerative cells to a culture medium, the culture medium designed to enable growth and maintenance of the regenerative cells; allowing secretion of beneficial factors from the regenerative cells into the medium so as to form a conditioned medium; removing from the conditioned medium an extract that is free of whole cells; and applying the extract and/or components of the extract topically to a portion of a skin of a human to foster growth of skin cells.

In another aspect of the invention, there is presented a method of assisting in growing human hair on a human scalp, comprising applying topically to a human scalp a cell-free extract of a conditioned medium, the conditioned medium made by culturing mesenchymal stem cells in a culture medium for between approximately 4 hours to approximately 3 days so as to allow secretions of the mesenchymal stem cells to enter and condition the culture medium.

In another embodiment of the present invention, there is a method of improving a quality of human hair that is growing on a human scalp, comprising (a) culturing regenerative cells in a culture medium for between approximately 4 hours to approximately 3 days so as to allow secretions of the mesenchymal stem cells to enter and condition the culture medium so as to form a conditioned medium; and (b) applying topically to the human hair a cell-free extract of a conditioned medium and/or components of the cell-free extract.

In accordance with another embodiment of the present invention, there is a composition for treating dermatological conditions and/or hair conditions, comprising a cell-free extract of a conditioned medium, the conditioned medium obtained by allowing a culture medium to culture regenerative cells and receive secretions of the regenerative cells for between approximately four hours and approximately three days, the culture medium conducive to growth and maintenance of the regenerative cells.

The present invention successfully addresses the shortcomings of the presently known skin and hair care compositions and methods by providing a method and composition that may more effectively and speedily grows new skin and/or hair cells and that may improve the quality of skin and hair to administer a therapeutic and/or cosmetic intervention. Furthermore, in contrast to the prior art, in which chemicals may be used that are harmful, the method and composition of the present invention may use the natural and healthy biochemistry of growth factors and other regenerative cells. In further contrast to prior art skin and hair care formulations and methods, the present invention exploits the paracrine effect to use the most beneficial ingredients secreted into a medium and extracted for use in a skin and hair care formulation.

The present invention discloses an innovative method and composition for skin care and hair care. Regenerative cells, as defined below, may be cultured to obtain conditioned media. An extract, which may be a supernatant and/or a cell-free extract of the conditioned medium may be obtained and applied topically onto a human's hair or skin to foster growth of skin cells. The extract may also be used together with other reagents in a skin or hair care formulation.

### DESCRIPTION OF THE PREFERRED EMBODIMENTS

The principles and operation of a method and composition for skin and hair care according to the present invention may be better understood with reference to the drawings and the accompanying description.

As a matter of definition, the term “regenerative cells” is intended to mean any combination in any proportion of the following: mesenchymal stem cells, endothelial progenitor cells, hematopoietic stem and progenitor cells, monocytes, macrophages, keratinocytes, fibroblasts, and any other cell type, excluding embryonic stem cells, that produces or secretes growth factors or cytokines or regulatory factors. It should be noted that the regenerative cells may be obtained from a single donor, or pooled from multiple donors. The regenerative cells may be used immediately when donated or may be used only after being frozen for a time.

The mesenchymal stem cells and endothelial progenitor cells or their mixtures may be obtained from different tissues in the adult body such as bone marrow, fat blood, umbilical cord blood, or from Wharton's Jelly or placenta or amniotic fluid, from normal deliveries, in which other cord components such as cord blood stem cells or cord serum or plasma have been removed and used for other applications, or from frozen cord blood units stored in public or family cord blood banks. Stem cells and progenitor cells may also be obtained from bone marrow or peripheral blood or mobilized peripheral blood, or cord blood or menstrual blood or fat tissue, or any tissue in the body that may be an efficient source for stem, progenitor or regenerative cells. In some embodiments, such as where the regenerative cells are obtained from an entire umbilical cord without previous removal of blood vessels or any other tissue, mesenchymal stem cells may be mixed together with other types of stem and progenitor cells such as endothelial progenitor cells and fibroblasts.

The “beneficial factors” that may be secreted from the regenerative cells, for example from the mesenchymal stem cells, is intended to refer to factors that have a beneficial effect on other cells of the human body. The following is a not-necessarily exhaustive list of examples of “beneficial factors”: growth factors, regulatory factors, hormones, enzymes, lymphokines and peptides. The beneficial factors enrich the culture medium so as to enable it to become a conditioned medium.

The culture medium may be a composition such as tissue culture that fosters the growth and maintenance of the regenerative cells used with the medium. The culture medium may include antibiotics, amino acids, water, salts and other components, reagents, serum, plasma or growth factors that are required for cell growth. One example of a culture medium used for the present invention is RMPI. For example, RPMI-1640 nedium has been used for the culture of human normal and neoplastic leukocytes and with supplementation supports growth of many types of cultured cells, including fresh human lymphocytes. Another example of a culture medium is DMEM which may be obtained from a number of companies including at www.invitrogen.com. A further example of a culture medium may be human blood plasma supplemented with components such as vitamins, amino acids, antibiotics, salts, water, etc.. The culture medium may also have been induced further for production of cytokines or growth factors using various induction methods such as exposure to hypoxia, or serum deprivation or various stimulating reagents. The culture medium may also include serum, such as human blood serum and/or fetal calf serum. Purely by way of illustration, the total volume of the culture medium may be made up of between approximately 5% and approximately 15% blood serum, which may be human or fetal calf blood serum. In one embodiment, the blood serum is 10% of the volume of the culture medium.

The extracts of the cultured medium may be from primary passages or from subsequent passages or cell lines derived from them. So for example, the extract or the supernatant may be taken from the primary passage or from a subsequent passage and either may be used in the skin or hair care formulation and method. Furthermore, in introducing the extracts from the conditioned medium into cosmetic formulations for skin care and hair care, the extracts may be used without further treatment or processing or without being fractionated or without having components thereof isolated therefrom

The present invention may be a composition for treating dermatological conditions and/or hair conditions, comprising a cell-free extract of a conditioned medium, the conditioned medium obtained by allowing a culture medium to receive secretions from regenerative cells for between approximately four hours and approximately three days. In one formulation, the regenerative cells may be selected from the group consisting of mesenchymal stem cells, endothelial progenitor cells, hematopoietic stem cells, progenitor cells, monocytes, macrophages, keratinocytes and fibroblasts. In one embodiment, the secretions include growth factor, regulatory factors, hormones, enzymes, lymphokines and peptides.

The composition of the present invention may also include placenta extracts, extracts from Wharton's jelly, or amniotic fluids or components extracted fro these tissues, or cord blood plasma, cord blood serum or components derived from them.

In accordance with one embodiment, the present invention is a method. This method may include a first step of culturing regenerative cells in a culture medium by adding regenerative cells (which may be mesenchymal stem cells and/or other regenerative cells) to a culture medium, where the culture medium is designed to enable growth and maintenance of the regenerative cells. Because regenerative cells have extensive paracrine effect they may secrete a wide variety of cytokines and growth factors into cultured medium. Accordingly, the method may further include a step of allowing the secretion of “beneficial factors” from the regenerative cells into the medium to form a conditioned medium. In certain embodiments, growth factor or blood serum may be added to the culture medium or to the conditioned medium.

The method may include a further step of removing from the conditioned medium an extract that is free of whole cells. The method contemplates removing the extract in at least two different ways. In accordance with the first way, removing the extract involves removing a supernatant of the conditioned medium. In a second approach, removing the extract involves extracting a cell-free extract from the cultured cells of the conditioned medium. In either case, whole cells [and basic cellular components] are not included.

The method may further include a step of applying the extract, and/or isolating and applying components of this extract, topically onto a portion of the skin or in the case of therapeutic use, onto an affected area of the skin, of a human for either therapeutic or cosmetic purposes, such as to foster growth of skin cells or hair cells. The method may also include a step of adding known cosmetic or therapeutic skin care formulations to the extract, for example to make it more effective, more stable or for other reasons, and only afterwards applying the total formula to the skin.

The present invention also embodies a method of improving the quality of human hair that is growing on a human scalp. This embodiment of the method may include a step of culturing regenerative cells in a culture medium, for example for between approximately 4 hours to approximately 3 days, so as to allow secretions of the regenerative cells, which may be mesenchymal stem cells, to enter and condition the culture medium so as to form a conditioned medium. This method may further include a step of applying topically to the human hair a cell-free extract of a conditioned medium and/or components of the cell-free extract. The step of applying topically to the human hair may involve smearing onto the hair either at the scalp or onto the hair at points away from the scalp. In some case, applying topically to the human hair may involve including the cell-free extracts in a shampoo and shampooing the hair with that shampoo.

By controlling the concentrations and formulations of the regenerative cells and the culture medium, one may custom tailor the secretions into the culture medium and custom tailor the composition of the conditioned medium and custom tailor the final product, the extract, used topically on the human skin or human hair, to achieve particular cosmetic and therapeutic purposes.

It is contemplated that the culture medium and/or the extracts of the culture medium can be tested for various contaminating agents or toxic materials, and/or tested for efficacy in various in vitro and in vivo models of skin and tissue regeneration or disease models.

In certain embodiments, an apparatus such as a bandage or other carrier may incorporate the composition of the present invention or components thereof. The bandage or other carrier material may be used by being applied to the skin or on a skin injury, or to skin diseases for cosmetic or therapeutic applications.

It is also contemplated that the present invention to use the conditioned medium, extracts of the conditioned medium, components of the extracts of the conditioned medium to be administered to a subject in need of food supplement, as well as in combinations with other food supplements or nutritional components.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A method, comprising:
adding regenerative cells to a culture medium, the culture medium designed to enable growth and maintenance of the regenerative cells;
allowing secretion of beneficial factors from the regenerative cells into the medium so as to form a conditioned medium;
removing from the conditioned medium an extract that is free of whole cells; and
applying the extract and/or components of the extract topically to a portion of a skin of a human to foster growth of skin cells.

2. The method of claim 1, further comprising adding the extract to a topical skin composition before applying the extract topically to the skin.

3. The method of claim 1, wherein removing the extract involves removing a supernatant of the conditioned medium.

4. The method of claim 1, wherein removing the extract involves extracting a cell-free extract from cultured cells of the conditioned medium.

5. The method of claim 1, further comprising adding growth factor before removing the extract.

6. The method of claim 1, wherein the culture medium includes between approximately 5 and approximately 15% of blood serum, the blood serum comprising fetal calf serum or human blood serum.

7. The method of claim 1, wherein the step of allowing the secretion of beneficial factors from the regenerative cells into the medium to form a conditioned medium is allowed to occur for between approximately four hours and approximately three days.

8. The method of claim 1, wherein the regenerative cells are mesenchymal stem cells.

9. A method of assisting in growing human hair on a human scalp, comprising:
applying topically to a human scalp a cell-free extract of a conditioned medium, the conditioned medium made by culturing mesenchymal stem cells in a culture medium for between approximately 4 hours to approximately 3 days so as to allow secretions of the mesenchymal stem cells to enter and condition the culture medium.

10. A method of improving a quality of human hair that is growing on a human scalp, comprising:
(a) culturing regenerative cells in a culture medium for between approximately 4 hours to approximately 3 days so as to allow secretions of the mesenchymal stem cells to enter and condition the culture medium so as to form a conditioned medium; and
(b) applying topically to the human hair
(i) a cell-free extract of a conditioned medium and/or
(ii) components of the cell-free extract.

11. The method of claim 10, wherein topically applying includes smearing onto the hair.

12. The method of claim 10, wherein topically applying involves including the cell-free extracts in a shampoo and shampooing the hair with the shampoo.

13. The method of claim 10, further comprising extracting a supernatant of the conditioned medium to create the cell-free extract.

14. A composition for treating dermatological conditions and/or hair conditions, comprising:
a cell-free extract of a conditioned medium, the conditioned medium obtained by allowing a culture medium to culture regenerative cells and receive secretions of the regenerative cells for between approximately four hours and approximately three days, the culture medium conducive to growth and maintenance of the regenerative cells.

15. The composition of claim 14, wherein the regenerative cells are selected from the group consisting of mesenchymal stem cells, endothelial progenitor cells, hematopoietic stem cells, progenitor cells, monocytes, macrophages, keratinocytes and fibroblasts.

16. The composition of claim 14, wherein the secretions are selected from the group consisting of enzymes, hormones, growth factor, regulatory factors and lymphocytes.
